(19) United States
(12) Patent Application Publication
(10) Pub. No.: US 2024/0033409 A1
(43) Pub. Date: Feb. 1, 2024

(54) MEMBRANE GAS EXCHANGER

(71) Applicant: FRESENIUS MEDICAL CARE DEUTSCHLAND GMBH, Bad Homburg (DE)

(72) Inventor: Jürgen KLEWINGHAUS, Oberursel (DE)

(73) Assignee: FRESENIUS MEDICAL CARE DEUTSCHLAND GMBH, Bad Homburg (DE)

(21) Appl. No.: 18/255,950

(22) PCT Filed: Dec. 3, 2021
(86) PCT No.: PCT/EP2021/084166

§ 371 Date: Jun. 5, 2023

(30) Foreign Application Priority Data
Dec. 7, 2020 (DE) 10 2020 132 472.3

(51) Int. Cl. A61M 1/16

(52) U.S. Cl. CPC A61M 1/1698

(57) ABSTRACT

The present invention relates to a membrane gas exchanger having a housing in which a first chamber and a second chamber as well as a membrane are arranged, wherein the membrane is gas permeable and liquid impermeable and separates the first chamber and the second chamber from one another, wherein the first chamber forms the blood side and the second chamber forms the gas side of the membrane gas exchanger, and wherein the first chamber has a blood inlet and a blood outlet, and wherein the second chamber has a gas inlet and a gas outlet, and wherein the blood inlet, the blood outlet, the gas inlet, and the gas outlet are arranged at the housing, wherein the housing is the housing of a dialyzer, and wherein a first adapter is provided that has an inlet and at least two outlets, with the inlet being connected to the gas outlet of the housing.

The present invention relates to a membrane gas exchanger having a housing in which a first chamber and a second chamber as well as a membrane are arranged, wherein the membrane is gas permeable and liquid impermeable and separates the first chamber and the second chamber from one another, wherein the first chamber forms the blood side and the second chamber forms the gas side of the membrane gas exchanger, and wherein the first chamber has a blood inlet and a blood outlet, and wherein the second chamber has a gas inlet and a gas outlet, and wherein the blood inlet, the blood outlet, the gas inlet, and the gas outlet are arranged at the housing.

Such membrane gas exchangers are known from the prior art.

They are used, for example, in devices for the extracorporeal decarboxylation of blood (ECCO2R) or also in devices for the extracorporeal membrane oxygenation of blood (ECMO). These devices are used when artificial respiration is required or when respiratory insufficiency is present so that a sufficient gas exchange (oxygen supply of the blood and CO₂ removal from the blood) with the blood via the lung is not possible.

In extracorporeal decarboxylation and extracorporeal membrane oxygenation, the blood is conducted through an extracorporeal circuit and through the blood side of the membrane gas exchanger located therein and in so doing carbon dioxide is removed from the blood through the gas outlet of the membrane gas exchanger and oxygen is supplied through the gas inlet of the membrane gas exchanger. The device pumps blood continuously through the membrane gas exchanger, that replaces the gas exchange in the lung, by means of a blood pump located in the extracorporeal circuit The blood prepared in this manner is then returned from the extracorporeal circuit to the patient. As the blood flow drops, the proportion of the oxygenation becomes smaller and the proportion of the decarboxylation becomes larger. The oxygen supply can also take place directly with a low blood flow, via mask respiration, a nasal oxygen tube, or a tube in the trachea.

A disadvantage of known membrane gas exchangers comprises the comparatively high manufacturing costs that are due to the high costs for the membrane material and due to the fact that the membrane gas exchangers are only produced in relatively small volumes and thus only a small degree of automation is present.

It is the underlying object of the present invention to further develop a membrane gas exchanger of the initially named kind such that it can be manufactured inexpensively.

This object is achieved by a membrane gas exchanger having the features of claim 1. Provision is accordingly made that the housing of the membrane gas exchanger is the housing of a dialyzer and that a first adapter is provided that has an inlet and at least two outlets, with the inlet being connected to the gas outlet of the housing.

Provision is thus made in accordance with the invention that the housing of a dialyzer is used for a membrane gas exchanger. The production means of dialyzer production can thus be made use of with respect to the housings and optionally also to the membranes that are made liquid tight, e.g. by a coating, with respect to conventional dialyzer membranes.

Unmodified dialyzer housings are preferably used for the membrane gas exchanger in accordance with the invention. These housings or their inlets or outlets for dialyzate are standardized for installation in the environment of dialysis, but do not meet the demands on membrane gas exchangers. A first adapter that has an inlet and at least two outlets is provided for the adaptation to the demands on membrane gas exchangers, with the inlet being connected to the gas outlet of the housing.

The inlet of the first adapter is thus connected to the gas outlet of the membrane gas exchanger. The gas outlet is the dialyzate outlet or dialyzate inlet of a dialyzer used for dialysis.

If a membrane gas exchanger having a standard housing of a dialyzer is used, it is important that the gas outlet is in no way closed. If the gas outlet is closed, this can result in a reduction in the elimination performance of CO₂ and in an unwanted gas transfer to the blood side, i.e. into the first chamber of the membrane gas exchanger, due to the pressure increase in the second chamber, which can substantially endanger the patient, for example by gas bubbles in the bloodstream.

Provision is made against this background that the first adapter has at least two outlets so that the likelihood of a closure of the gas outlet of the membrane gas exchanger is substantially reduced. A conventional dialyzer housing is made safer on the use as a housing of a membrane gas exchanger due to the use of the first adapter. At least one outlet can be designed as a non-planar surface having dimples to increase safety. A closing of the opening by pressing on a planar surface can thus be prevented.

It is possible to profit from the substantial volumes of dialyzers with regard to production costs by the use of a conventional dialyzer housing as the housing for a membrane gas exchanger since the production facilities for the production of the housings do not have to be modified.

The first adapter includes the functionality that the gas outlet cannot be closed.

The first adapter is preferably releasably connected to the gas outlet. It can, for example, be latched thereto or connected by a snap-on closure.

In a preferred embodiment, the first adapter is plugged onto the gas outlet.

Provision can be made, as with a conventional dialyzer that the membrane is present as a hollow fiber bundle whose ends are received in molding compounds such as is known from conventional dialyzers. Provision is preferably made here that the inner space of the hollow fibers forms the first chamber, i.e. the blood chamber, and the space surrounding the hollow fiber bundle forms the second chamber, i.e. the gas side.

The membrane can have a base body and a liquid permeable coating located thereon so that it is prevented that liquid components of the blood enter into the second chamber.

The membrane gas exchanger preferably has a gas inlet that is connected to a second adapter. The gas inlet serves, for example, the supply of pure oxygen, air, or of a gas mixture as a flushing gas.

The second adapter is also preferably releasably connected to the gas inlet and is preferably plugged thereon. It is furthermore conceivable that the second adapter is differently designed than the first adapter. It must be ensured by means of the first adapter, via which gas is discharged from the second chamber, that this gas outflow is not closed. This requirement is not present for the second adapter by means of which gas is introduced into the second chamber.

The second adapter can have an inlet and an outlet, with the outlet of the second adapter being connected to the gas inlet.

Provision is preferably made that a retaining section is present in the region of the inlet of the second adapter by means of which retaining section a hose can be fixed to the inlet. The retaining section is a pine section, a saw tooth section, etc., for example.

The first adapter can have a quick-closing connection for coupling to the gas outlet so that it can be snapped onto the gas outlet of a dialyzer in an easily operable manner. The same can apply accordingly, alternatively or additionally, to the second adapter.

It is conceivable that the first adapter has a gas sample outlet. A measuring glass for determining the gas components in the outflowing gas, e.g. for determining the partial CO₂ pressure can thus be acquired.

It is also conceivable that the first adapter has an outflow for condensed water. A connection, e.g. a Luer connector, can thus be provided to provide a capturing possibility, e.g. an empty flushing bag for condensed water.

The present invention further relates to a device for extracorporeal membrane oxygenation or for extracorporeal decarboxylation or for another gassing or degassing of blood using an extracorporeal blood circuit, wherein the extracorporeal blood circuit is connected in accordance with the present invention, to the blood inlet and blood outlet of a membrane gas exchanger.

The second adapter of the membrane gas exchanger is preferably connected to an oxygen source for the purpose of blood oxygenation. It can alternatively or additionally be connected to a flushing gas source or to another gas source.

It is further conceivable that the first adapter of the membrane gas exchanger is connected to a CO₂ discharge so that a sufficient decarboxylation of the blood is possible.

The present invention further relates to the use of a dialyzer housing for a membrane gas exchanger in a device for extracorporeal membrane oxygenation and/or for extracorporeal decarboxylation or for another gassing or degassing of blood.

Further details and advantages of the invention will be explained in more detail with reference to an embodiment shown in the drawing. There are shown:

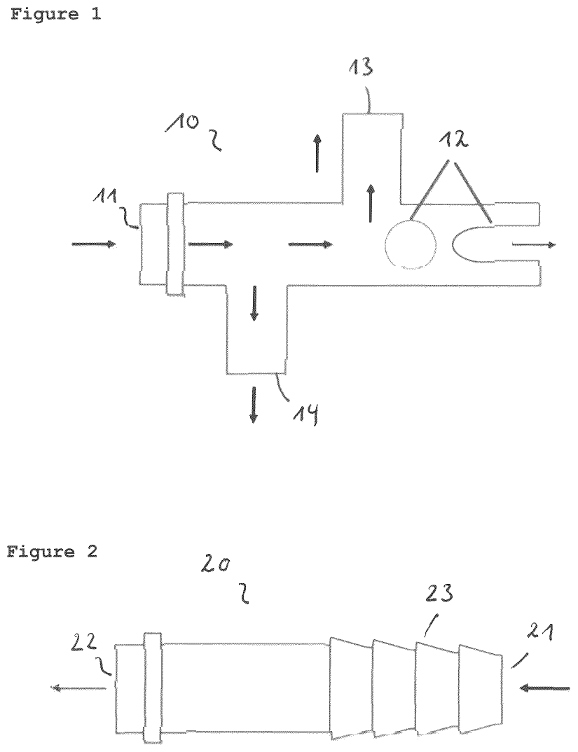

FIG. 1: a schematic view of the first adapter;

FIG. 2: a schematic view of the second adapter; and

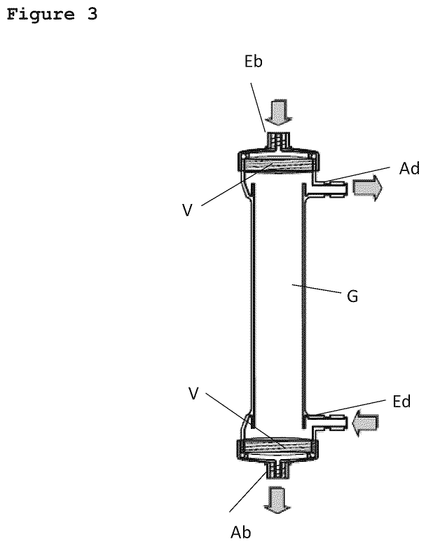

FIG. 3: a schematic view of a housing of a known dialyzer.

In the embodiment, the housing G of a conventional dialyzer is used such as is shown by way of example in FIG. 3. It has an inlet Eb and an outlet Ab for blood and an inlet Ed and an outlet Ad for dialyzate, A hollow fiber bundle, not shown, whose inner spaces are in fluid communication with the blood side and thus also with Eb and Ab and that is surrounded by a space that is in turn in fluid communication with Ed and Ad, extends between the molding compounds V.

The outlet Ad or the inlet Ed for dialyzate is used in accordance with the invention as the gas outlet of a membrane gas exchanger that in turn serves the gassing and/or degassing of blood in an extracorporeal circuit of an ECMO or ECCO2R device. As can be seen from FIG. 3, the outlet Ad and the inlet Ed are each formed as stubs projecting from the housing G.

The housing G is preferably cylindrical and has the blood inlet Eb and the blood outlet Ab at its front faces and the inlet Ed and outlet Ad for dialyzate or (in the case of a use as a membrane gas exchanger) the inlet and outlet for gas at its jacket surface. All the inlets and outlets are preferably designed as stubs that project from the housing G, as can be seen from FIG. 3.

The first adapter 10 is plugged in accordance with FIG. 1 onto the gas outlet tub Ad and is secured there by means of a snap-on closure connection.

The first adapter 10 has an inlet 11 for the gas flowing out of the housing (not shown) and a plurality of gas outlets 12 through which the gas exits the first adapter 10. As can further be seen from FIG. 1, the first adapter 10 has a gas sample outlet 13 and furthermore an outlet 14 for discharging condensed water in the second chamber. The likelihood of a closure of the membrane gas exchanger on the gas outlet side and thus the risk for the patient is reduced by the plurality of gas outlets 12.

It can furthermore be seen from FIG. 1 that the gas outlet 12 shown on the right is not formed as a planar, e.g. circular surface, but rather has cutouts extending to the planar front face in the jacket surface, whereby the closing of this outlet by a planar surface can be prevented.

The second adapter 20 in accordance with FIG. 2 is connected to the gas inlet stub Ed of the housing G, with the former preferably likewise being secured at the gas inlet stub Ed by a snap-on closure. The second adapter 20 has an inlet 21 for e.g. a flushing gas or for oxygen and an outlet 22 that is plugged onto the gas inlet stub Ed of the membrane gas exchanger. As can be seen from FIG. 2, the inlet 21 of the second adapter 20 is surrounded by a pine section 23 so that a hose or the like can be fixed to the second adapter 20 in a comparatively simple manner.

1. A membrane gas exchanger having a housing in which a first chamber and a second chamber as well as a membrane are arranged, wherein the membrane is gas permeable and liquid impermeable and separates the first chamber and the second chamber from one another, wherein the first chamber forms the blood side and the second chamber forms the gas side of the membrane gas exchanger, and wherein the first chamber has a blood inlet and a blood outlet, and wherein the second chamber has a gas inlet and a gas outlet, and wherein the blood inlet, the blood outlet, the gas inlet, and the gas outlet are arranged at the housing, characterized in that the housing is the housing of a dialyzer; and in that a first adapter is provided that has an inlet and at least two outlets, with the inlet being connected to the gas outlet of the housing.

2. A membrane gas exchanger in accordance with claim 1, characterized in that the first adapter is releasably connected to the gas outlet.

3. A membrane gas exchanger in accordance with claim 1, characterized in that the first adapter is plugged onto the gas outlet.

4. A membrane gas exchanger in accordance with claim 1, characterized in that the membrane is present as a hollow fiber bundle.

5. A membrane gas exchanger in accordance with claim 1, characterized in that the membrane has a base body and a liquid impermeable coating located thereon.

6. A membrane gas exchanger in accordance with claim 1, characterized in that the membrane gas exchanger has a gas inlet that is connected to a second adapter.

7. A membrane gas exchanger in accordance with claim 6, characterized in that the second adapter is releasably connected to the gas inlet and is preferably plugged onto the gas inlet.

8. A membrane gas exchanger in accordance with claim 6, characterized in that the second adapter is differently designed than the first adapter; and/or in that the second adapter has an inlet and an outlet, with the outlet being connected to the gas inlet, and with provision preferably being made that a retaining section is present in the region of the inlet by means of which retaining section a hose can be fixed to the inlet.

9. A membrane gas exchanger in accordance with claim 1, characterized in that the first adapter and/or the second adapter has/have a quick-closure connection for coupling to the gas outlet or to the gas inlet.

10. A membrane gas exchanger in accordance with claim 1, characterized in that the first adapter has a gas sample outlet.

11. A membrane gas exchanger in accordance with claim 1, characterized in that the first adapter has an outflow for condensed water.

12. A device for extracorporeal membrane oxygenation or for extracorporeal decarboxylation or for another gassing or degassing of blood using an extracorporeal blood circuit that is connected to the blood inlet and blood outlet of a membrane gas exchanger in accordance with claim 1.

13. A device in accordance with claim 12, characterized in that the second adapter of the membrane gas exchanger is connected to an oxygen source or to a flushing gas source.

14. A device in accordance with claim 12, characterized in that the first adapter of the membrane gas exchanger is connected to a CO₂ discharge.

15. A use of a dialyzer housing for a membrane gas exchanger in a device for extracorporeal membrane oxygenation and/or for extracorporeal decarboxylation or for another gassing or degassing of blood.
